SEQUENCE LISTING (6 sequences)

SEQ ID NO: 1 - protein, 3067 aa, Portunus trituberculatus
MPGHVKKSTGPDPDPSEWLYISNEMKMKDATKPYDPKKSCWVPDPKEGYLEGLIQGTKGEKITVMAGGETKEYKKDQVHQVNPPKYEKCEDMSNLTYLNDPSVLYNLKARYVNKLIYTYSGLFCIVVNPYKRYPIYTNRTVKIYQNKRRNEVPPHLFAISDGAYSAMLQATCLLSNDIYDYGYVSQGKVTVPSIDDGEDMQFCHDAFDILGFTKTEIENVYKITAVVMHMGNMKFKQKGREEQAEADGKEHGAKCATLLGCDVEDMYLAITKPKIKVGTEFVAKGMNVNQCNYSIGAMAKALFDRVFKFIVSKCNETLETGLKRASFIGVLDIAGFEIFDYNGFEQICINFCNEKLQQFFNHHMFVLEQEEYKKEGINWVFVDFGMDLQACIELFEKKMGLLSILEEESMFPKATDKSFAEKLNANHLGKTPCFIKPKPPKAGQTEAHFAIVHYAGTVSYNLSGWLEKNKDPLNDTLVDVLKKSSNELAVSIFADHPGQSGGDDKKGGGRGAKSGSFKTVSSSYREQLNNLMRTLNATHPHFIRCIVPNETKSPGVVDAGLVMHQLTCNGVLEGIRICRKGFPNRIPYSDFKHRYLILAPKEMAEAKTDKNMAQACFTKAGLDEESYRLGNTKVFFRAGILGRMEELRDDRICLLLAWLQAWVRGYISRKTYSKLQKQRISLLVVQRNIRKYMKMRTWKWYGMWMVLKPTLHVVSIEDELRNLEEKAEKAEKEYEEMVKKHQELEKVNSGLTSEKNELADALNSSKDHPHFICIPFSENKQEAQKRLRNEEDARNKLQQSNKKTEQEVDSLRKDMEDLEQAIQKSEQDKATKDQQIHNMNEEVAHQEELINKINKEKKHLQECTQKTSEDLQNLEDKCNHFINIKAKLESNLDEVEDTLEREKKLRNDVEKAKRKVEGDLKLSQEAVADLERNQKEIEATILRKEKEHSALVSKIEEEQAQYTKLQRQIKEQQTRVEDLEHEAEHERQARAKAEKAKTTLAQELSELADQLDEAGGATAAQIELNKKREAELAKLRRDLEEINIQHEAALSQLRKKHNDTINEMSEQIDHHNKLKARLEKDKDTLKRDAEDAKAALDSLVHEKAAAEKTLKQIQQQIADHSGKLDEMNRTLNDFDSTKKKLSADNNDLQRQIEEAESMISQLSKMKLSLTNQLEDHRKLADEESRDRATLLGKCRNLEHDIESLREQLEIESEAKGDVQRMLSRASAEAQMWRSKYENEGVARVEEIEASRLKLAARLDEAESQIEQLNVKNVSLEKAKQRLLTDIEETQIAAERAKTIADSAEKKQKHYDRILSEWKLKVDDLSSNLDSSQKECRNYSTEVFRVKAAYEENLEQLDSIRRENKNLNDEIKDLVDQIGEGGRAYHEIEKSVKRLDIEKEELQAALEEAEAALEQEENKVLRGQLELSQMEYKDMELKVEEQHRIASEFRDQYNMAERRGNALQGELEESRSLLEQSDRARRQAESDLADANDQLNELGTQCSDMSLSKRKVEGEIQTLQTDLDEMLSEAKNSEEKAKKAMVDAARLADELRSEQEHAQNQEKMRKGLDVSLKELQARLEESEMNAQKAIKNAVAKLEGRVRELEVNLTEESRHYSDAQKNLRKCERRIKELTFQAEEDKKNHEKMQDLVDKLQQKMKTYKRQIEEAEEIAALNLANGESGAGKTENTKKVLSYFANVGATTGKKKEGEKEKPFREQYAGAERRANQINSELEESRTLLEQSDRGRRQAESNLLEANDQVSDLNAQYGALSVAKKKLEGEYQALQAEEDKKNHERMQDLVDGLQQKIKTYKRQIEEAEEIAALNLANGESGAGKTENAKKLVSYFAKNAASASENTEDSEKPSLADRIGHSNIILEAFGNAKTARNDNSSRFKFQQFYNYCNFVKVQEEYKKEGIEWTPPEVNLDLQPSIDFFEKKLGFMPIMDEESMSPKGTEKTFLEKLNANHLGKTPTFIKPKPPKPDQPEAHFAIVHYAGTVNYDLTGWLEKNKDPLNESTVDLLKTSSNELVVAIFTDPPPKPAEAETEEAKEGKEAKEAKEAKEVKEVKVKGKPLARGKGAAKGKDAGKDSAKEAEVIYKTASSGYREQLNNLIKTLNATHPRFIRCILPNETKSPVVQRNLRKYIRIHAWKWQGLLENIRPLLNITKIEDEMKVLAEKTAKALDDLEKETKLRKNLEIENMKLQEDKYDLMATLDSSKADIKEFLEKQAKLQSQKTDLEMQLEDTNKLLQKEEEGRTAVTQTKKKLEYNLHEIRQDLDDMELAMHKAQHAEAAKEQVIQGLKEEINSQEELIKKLKKEKKHLQEMNQKTAEDNQNIEDKCTHLNILKGNLETHLEKLEGFLDHEKKLRSAAEREKRKAESDLKMSQAAVSEFNRTRKELEQAIDRKNKEVSALTIKVEEDYQQITRLSKQIKDMEKRQDEQDAEILQERQGRSAAETAKVALTHEVTELSNRLSEASAYHNLEHDTGKIKEKLDEECDNGKNLQRLISKAYDEAAMWRSKYEIEGVTRVEELEASRQKLSCRLEETEGKIDQLNAKNINLEKIKQRTAAELFEIQGELHRTKALVDAAEEKQKNFDSAVEEWKVKVDNLTSELEASRKEVRKHGSETSRVNALYKETLVQVDSLRVENKGLAQEVKSLNEEVSSKTDVLHKLQLAFKRLKGEKDGLQGDLEDAEAALDQEEVKVQRGQVELNQIKQEMERRVQEKEEEFEKSRKFYMSSVDSLRVSLETEVKDKAEALYLKKKLESEVQQLQGTIQRAKEANNDLLKAKERAEKENEEQQVRLEEAEHIISQQREQVSTIERRIASLMGELEEAHTLLEQADRSRRSADVNLTDANDYIRKLTAENENLTSTKERLEEELVCMQNEFEDMIKEARGSDDKAKRVAMEAARLGEELRGEQDNAKAQEKLRKDLEVSLREMQARLDADSSNQKVGRAAFEKMEARVHDLETQLEQETIRYAESQKNFRRSERRVKELTLRTEEDQKNHESMQDLVDKLQSKIKGYKRQMEEAEEIAALNLAKYRKAQQELEEFEENASLRSKVTRMEAPSVVV

SEQ ID NO: 2 - protein, 19 aa, synthetic construct
STGPDPDPSEWLYISNEMK

SEQ ID NO: 3 - protein, 5 aa, synthetic construct
ALFDR

SEQ ID NO: 4 - protein, 5 aa, synthetic construct
SGSFK

SEQ ID NO: 5 - protein, 9 aa, synthetic construct
ASAEAQMWR

SEQ ID NO: 6 - protein, 4 aa, synthetic construct
LSCR

(19) United States
(12) Patent Application Publication
(10) Pub. No.: US 2026/0008826 A1
(43) Pub. Date: Jan. 8, 2026

(54) UMAMI PEPTIDE DERIVED FROM PORTUNUS TRITUBERCULATUS AND APPLICATION THEREOF

(71) Applicant: OCEAN UNIVERSITY OF CHINA, Qingdao City (CN)

(72) Inventors: XIANGZHAO MAO, Qingdao City (CN); YANMEI REN, Qingdao City (CN); JIAN'AN SUN, Qingdao City (CN); QINGZE SUN, Qingdao City (CN); SHIXIN HUA, Qingdao City (CN)

(21) Appl. No.: 19/250,466

(22) Filed: Jun. 26, 2025

(30) Foreign Application Priority Data
Jul. 3, 2024 (CN) 202410881140.8

(51) Int. Cl. C07K 14/435

(52) U.S. Cl. CPC C07K 14/43509

(57) ABSTRACT

The present invention discloses an umami peptide derived from Portunus trituberculatus and an application thereof, and belongs to the technical field of bioactive peptides. An amino acid sequence of the umami peptide derived from the Portunus trituberculatus is SGSFK, as shown in SEQ ID NO. 4. The umami peptide derived from the Portunus trituberculatus is used as or in preparation of an umami agent. The present invention integrates virtual enzymatic hydrolysis, screening and docking technologies to extract the umami peptide from the Portunus trituberculatus. Through sensory evaluation and electronic tongue analysis, it has been verified that the umami peptide SGSFK in the present invention is more umami than monosodium glutamate at a same concentration, and has an umami threshold of 0.125 mg/ml. The present invention provides a new raw material basis for development of umami agents and condiments, and has good application prospects and important application value.

### REFERENCE TO AN ELECTRONIC SEQUENCE LISTING

The content of the electronic sequence listing (Sequence Listing.xml; Size: 9,459 bytes; and Date of Creation: Jun. 25, 2025) is herein incorporated by reference in its entirety.

### TECHNICAL FIELD

The present invention relates to an umami peptide derived from Portunus trituberculatus and an application thereof, and belongs to the technical field of bioactive peptides.

### BACKGROUND

Umami peptides are a class of small molecule peptides with umami characteristics. The umami peptides play an important role in formation of food flavor. Types of the umami peptides reported in the prior art are limited, and the discovery of a new umami peptide is one of the current research hotspots.

In recent years, virtual enzymatic hydrolysis, screening and docking technologies have been widely used in screening and prediction of bioactive peptides. The umami peptide interacts with umami receptors T1R1/T1R3 to activate the transmission of cascade signals from a taste system, making the brain perceive an umami taste, which provides an effective method for efficient screening of the umami peptide and revealing an interaction between the umami peptide and a receptor thereof.

Portunus trituberculatus, commonly known as a swimming crab, has tender meat and a delicious taste, and is a marine aquaculture crab with a great economic value. So far, there has been no report on umami peptides derived from the Portunus trituberculatus.

### SUMMARY

In view of the above-mentioned prior art, the present invention provides an umami peptide derived from Portunus trituberculatus and an application thereof, and belongs to the technical field of bioactive peptides.

The present invention is achieved by the following technical solution:

An amino acid sequence of the umami peptide derived from Portunus trituberculatus is SGSFK, as shown in SEQ ID NO. 4.

The umami peptide derived from the Portunus trituberculatus is used as or in preparation of an umami agent.

The present invention integrates virtual enzymatic hydrolysis, screening and docking technologies to extract the umami peptide from the Portunus trituberculatus. First, myosin (a sequence ID of MPC23537.1) with a high abundance in meat of the Portunus trituberculatus was selected from an NCBI database as an enzymatic hydrolysis target, and trypsin (an enzyme number in a KEGG database is 3.4.21.4), an endogenous protease with a high abundance in the Portunus trituberculatus, was selected as a hydrolase. Then, an amino acid sequence of the myosin was input into a program by using a BIOPEP-UWM online enzymatic hydrolysis program, the trypsin was selected for virtual enzymatic hydrolysis, and a theoretical peptide sequence was obtained. Subsequently, three combined sequence-based machine learning predictors were used sequentially to predict taste characteristics of the peptides, and peptides that satisfied both UMPred-FRL and TastePeptidesDM and had an umami taste and a PeptideRanker score greater than 0.5 were selected. Toxicity and water solubility of the selected peptides were tested by using two mini-programs of ToxinPretool and Innovagen tool, respectively. A molecular docking technology was used to simulate a molecular interaction between an umami receptor T1R1/T1R3 and the peptides, and potential umami peptides were screened out. Finally, the potential umami peptides were obtained through biological solid-phase synthesis, and sensory evaluation and electronic tongue verification were performed to obtain the umami peptides with a prominent umami taste.

The present invention uses machine learning for composite screening of the umami peptides in the Portunus trituberculatus, and as a result, three potential umami peptides were screened out, SGSFK, ALFDR, and ASAEAQMWR respectively. The interaction and surface force between the peptides and subunits of the umami receptor T1R1/T1R3 were analyzed, and it was found that the umami peptide SGSFK can bind to the subunits of the umami receptor T1R1/T1R3, which has a stable complex conformation. Furthermore, it has been verified from sensory evaluation and electronic tongue analysis that the umami peptide SGSFK has a richer umami taste than monosodium glutamate (MSG) at a same concentration, and has an umami threshold of 0.125 mg/ml; while the ALFDR and ASAEAQMWR had obvious saltiness-enhancing effects. The present invention provides a new raw material basis for development of umami agents and condiments, and has good application prospects and important application value.

The present invention uses an artificial intelligence method to perform online virtual enzymatic hydrolysis of proteins, so as to obtain peptides, and combines machine learning to perform composite screening of the umami peptides in the Portunus trituberculatus, which avoids time-consuming and labor-intensive disadvantages caused by step-by-step separation and purification, avoids the loss of polypeptides caused by cumbersome experimental steps, and greatly improves the screening efficiency, accuracy and repeatability of the umami peptides.

Various terms and phrases used in the present invention have the general meanings that are well known to those skilled in the art.

### BRIEF DESCRIPTION OF THE DRAWINGS

FIG. 1 is an electronic tongue taste radar map of each potential umami peptide, in which due to a small amount of acid and salt in a reference liquid during electronic tongue determination, tasteless points for sourness and saltiness are −13 and −6 respectively, while tasteless points for other taste indicators are 0;

FIG. 2 is an electronic tongue taste principal component analysis chart of each potential umami peptide, in which a horizontal axis PC1 represents a principal component 1 and a vertical axis PC2 represents a principal component 2;

FIG. 3 is a schematic diagram of electronic tongue taste significance analysis of each potential umami peptide, in which due to a small amount of acid and salt in a reference liquid during electronic tongue determination, tasteless points of sourness and saltiness are −13 and −6 respectively, tasteless points of other taste indexes are 0, and a, b, c, d, and e indicate that there are significant differences at the level of P<0.05;

FIG. 4 is a 3D structural diagram of an umami peptide SGSFK;

FIG. 5 is a ramachandran plot of an umami receptor T1R1/T1R3, in which ψ represents a rotation angle of a C—N bond on the left side of an α carbon in a peptide unit, and φ represents a rotation angle of a C—C bond on the right side of the α carbon;

FIG. 6 is a structural model of an umami receptor T1R1/T1R3;

FIG. 7 is schematic diagram of molecular docking between an umami receptor T1R1/T1R3 and an umami peptide SGSFK;

FIG. 8 is a schematic diagram of a receptor-ligand interaction force between an active site of an umami receptor T1R1/T1R3 and an umami peptide SGSFK.

### DETAILED DESCRIPTION OF THE EMBODIMENTS

The following further describes the present invention with reference to examples. However, the scope of the present invention is not limited to the following examples. Those skilled in the art can understand that various changes and modifications may be made to the present invention without departing from the spirit and scope of the present invention.

Instruments, reagents, and materials involved in the following examples, unless otherwise specified, are all conventional instruments, reagents, and materials already available in the prior art and may be obtained through regular commercial channels. Experimental methods, detection methods, etc. involved in the following examples, unless otherwise specified, are all conventional experimental methods and detection methods already available in the prior art.

### Example 1 Determination of Protein and Endogenous Enzyme in Portunus trituberculatus

The protein with a high abundance in the Portunus trituberculatus is myosin, and the myosin with a protein sequence ID of MPC23537.1 was selected from an NCBI database as an enzymatic hydrolysis target, and its amino acid sequence was shown in SEQ ID NO. 1. An endogenous proteolytic enzyme with a high abundance in the Portunus trituberculatus was trypsin, and the number of the enzyme in a KEGG database was 3.4.21.4.

### Example 2 Virtual Enzymatic Hydrolysis of Protein by Online Program

A BIOPEP-UWM online enzymatic program hydrolysis (https://biochemia.uwm.edu.pl/biopep-uwm/) was used, and “Bioactive peptides”, “ANALYSIS”, “ENZYME(S) ACTION”, and “for your sequence” were sequentially clicked. The amino acid sequence of the myosin determined in Example 1 was input into “Paste the sequence”, the trypsin determined in Example 1 was selected in “Enzyme ID”, and “View the report with results” was clicked for virtual enzymatic hydrolysis to obtain a theoretical peptide sequence.

Results: Virtual enzymatic hydrolysis was performed on the myosin (MPC23537.1) with the trypsin [EC: 3.4.21.4] by using the BIOPEP-UWM online enzymatic hydrolysis program, and 339 theoretical peptides were obtained.

### Example 3 Virtual Screening of Potential Umami Peptides

Three combined sequence-based machine learning predictors were used to predict taste characteristics of the theoretical peptides obtained in Example 2, including a UMPred-FRL, a PeptideRanker, and a TastePeptidesDM. Peptides that satisfied both the UMPred-FRL and the TastePeptidesDM and had an umami taste and a PeptideRanker score greater than 0.5 were selected for subsequent steps.

The URL of the UMPred-FRL is https://pmlabstack.pythonanywhere.com/UMPred-FRL.

The URL of the PeptideRanker is https://distilldeep.ucd.ie/PeptideRanker.

The URL of the TastePeptidesDM is http://tastepeptides-meta.com/TPDM.

Results: There were five peptides that satisfied both the UMPred-FRL and the TastePeptidesDM and had an umami taste and a PeptideRanker score greater than 0.5, namely: STGPDPDPSEWLYISNEMK, ALFDR, SGSFK, ASAEAQMWR, and LSCR. Among them, the STGPDPDPSEWLYISNEMK has a longer sequence and a too high synthesis cost, and the LSCR had been reported previously, so these two peptides were not used as subsequent research objects. For the ALFDR, SGSFK, and ASAEAQMWR, UMPred-FRL scores were 0.98, 0.841, and 0.969, respectively; PeptideRanker scores were 0.629, 0.620, and 0.666, respectively; and TastePeptidesDM scores were 0.994, 0.991, and 0.98, respectively. These three peptides were used as potential umami peptides for subsequent research.

An amino acid sequence of the STGPDPDPSEWLYISNEMK was shown in SEQ ID NO. 2. An amino acid sequence of the ALFDR was shown in SEQ ID NO. 3. An amino acid sequence of the SGSFK was shown in SEQ ID NO. 4. An amino acid sequence of the ASAEAQMWR was shown in SEQ ID NO. 5. An amino acid sequence of the LSCR was shown in SEQ ID NO. 6.

### Example 4 Verification of Toxicity and Water Solubility of Peptides

The toxicity and water solubility of the above three peptides were tested by using two tools of a ToxinPretool and a Innovagen tool respectively. Non-toxic and water-soluble peptides were selected as potential umami peptides for subsequent analysis.

The URL of the ToxinPretool is https://webs.iiitd.edu.in/raghava/toxinpred3/prediction.php.

The URL of the Innovagen tool is https://www.innovagen.com/proteomics-tools.

Results: The three peptides of the ALFDR, SGSFK and ASAEAQMWR were non-toxic and had good water solubility.

### Example 5 Solid-Phase Synthesis of Potential Umami Peptides

Sangon Biotech Co., Ltd. (Shanghai) was commissioned to synthesize the ALFDR, SGSFK and ASAEAQMWR by using an Fmoc solid phase method, with a purity not less than 95%.

### Example 6 Sensory Evaluation of Potential Umami Peptides

The sensory evaluation was conducted in a sensory analysis laboratory with a temperature of 23±2° C. and normal illumination. Prior to the evaluation, 10 sensory evaluation panelists (5 females and 5 males, aged between 20 and 30 years) received sensory training according to an international standard method (ISO 8586-1:2012) for training and supervising evaluators. Standard samples for umami, sweet, salty, bitter, and sour tastes were a monosodium glutamate solution (0.35%, g/ml), a sucrose solution (1.00%, g/ml), a sodium chloride solution (0.35%, g/ml), an L-isoleucine solution (0.25%, g/ml), and a citric acid solution (0.08%, g/ml), respectively. Ultrapure water was used to prepare each potential umami peptide into a 1 mg/ml of solution for the sensory evaluation of the panelists.

A triangle test (two cups of distilled water and one cup of potential umami peptide solution) was performed to determine a taste threshold. The potential umami peptide solution was gradually diluted with distilled water at a ratio (volume ratio) of 1:1 and marked, and submitted to the sensory evaluation panelists in the order of increasing concentration. A lowest concentration at which sensory evaluators could perceive the umami taste of the samples was recorded, and an average of the lowest concentrations recorded by all panelists was calculated as an umami peptide threshold.

Test results were shown in Table 1. As can be seen from Table 1, the SGSFK has the best effect.

TABLE 1
Umami threshold, salty threshold, and taste description of potential umami
peptides

| | Umami | Salty | |
|---|---|---|---|
| Potential umami | threshold | threshold | |
| peptides | (mg/ml) | (mg/ml) | Taste description |
| ALFDR | — | 0.125 | Obvious salty taste, prominent |
| | | | sour taste, and not obvious |
| | | | umami taste |
| SGSFK | 0.125 | — | Obvious umami and sweet |
| | | | taste, and slightly salty taste |
| ASAEAQMWR | — | 0.0625 | Heavily salty and sour taste, |
| | | | and slightly umami taste |

### Example 7 Determination of Potential Umami Peptides by Electronic Tongue

Ultrapure water was used to prepare each potential umami peptide into a 0.3 mg/ml of solution for electronic tongue analysis, and an MSG solution or a saline solution (with solute of NaCl) at a same concentration was used as references. Flavor characteristics of the potential umami peptides were determined by using an SA-402B taste analysis system (Insent SA402B, Tokyo, Japan). Data were collected for 4 times for each sample, and the first collected data was automatically deleted.

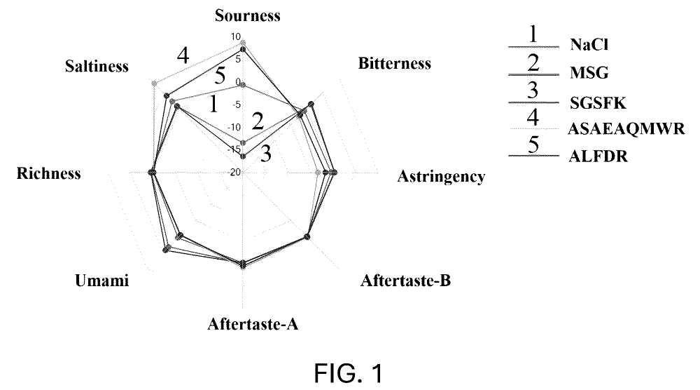

An electronic tongue taste radar map of each potential umami peptide was shown in FIG. 1, and it can be seen intuitively that the taste of each potential umami peptide was significantly different from that of MSG and NaCl.

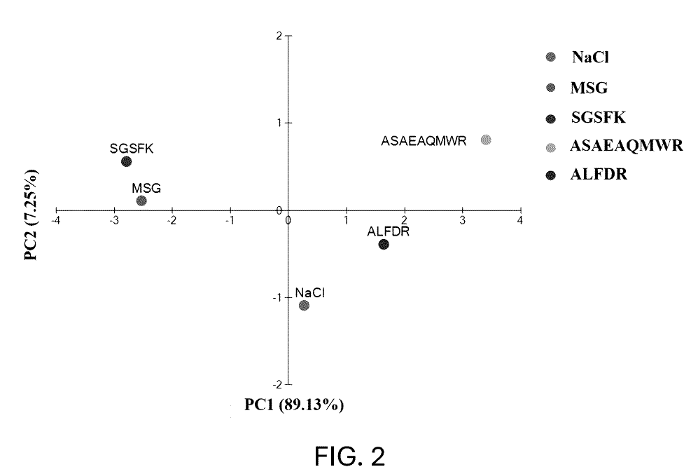

An electronic tongue taste principal component analysis chart of each potential umami peptide was shown in FIG. 2, it can be seen that the SGSFK was closer to taste components of the MSG, and it was determined that the SGSFK can be used as an umami peptide and an umami agent.

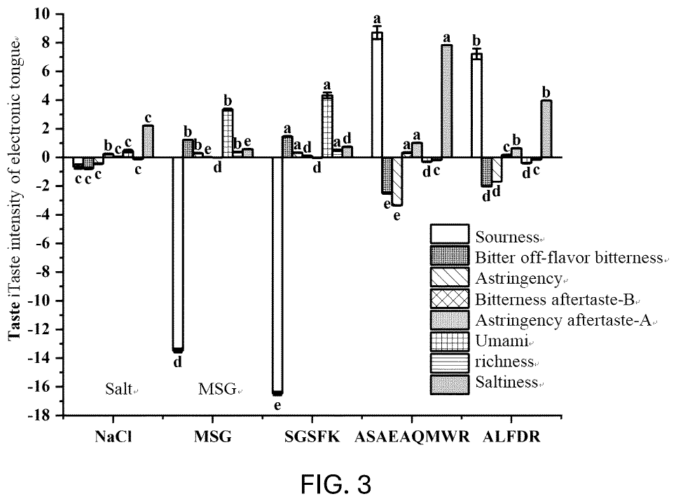

A schematic diagram of electronic tongue taste significance analysis of each potential umami peptide was shown in FIG. 3, and the significance analysis of each taste characteristic was performed. Generally speaking, if a difference in taste intensity between samples is less than 0.5, it can be considered that people cannot perceive the difference in taste. Although it was found from single-factor ANOVA significance analysis that saltiness, richness, bitterness, aftertaste-A and aftertaste-B of the SGSFK were significantly higher than those of the MSG, the difference could not be perceived by humans. Therefore, only the umami of the SGSFK was significantly better than that of the MSG, with the umami value of the SGSFK being 1.3 times that of the MSG. Compared with the SGSFK and MSG, the sourness, saltiness, and aftertaste-A of the ALFDR and ASAEAQMWR were significantly enhanced, and the aftertaste-B and umami were significantly reduced. The increase in the sourness and aftertaste-A may be affected by residual amino acids and sodium acetate in the peptide synthesis process. Therefore, the saltiness of the ALFDR and ASAEAQMWR were more prominent than that of the SGSFK. The saltiness values of the ASAEAQMWR and ALFDR were 3.53 and 1.79 times that of NaCl, respectively. Therefore, the ASAEAQMWR and ALFDR can be used as salty peptides and salty agents to achieve the effect of reducing salt.

Compared with the umami peptides derived from aquatic products reported in the prior art: the threshold of umami peptide (EEEVVEEVE, DEGDLDF) screened by Wang Yueqi et al. through fermentation of sea bass was 0.219-0.234 mg/mL, the threshold of scallop umami peptide (RPRVVR) extracted by Zhao Qin et al. was 0.25 mg/mL, and the threshold of oyster umami peptide (FNKEE) screened by Du Ming et al. was 0.38 mg/mL. The umami threshold of the umami peptide SGSFK in the present invention was lower (as low as 0.125 mg/mL). From some studies, 10 salt-increasing peptides had been screened from yeast protein, and the threshold was 0.13-0.50 mmol/L. In addition, from some studies, 16 salty peptides had been screened from Tilapia mossambica, and the threshold was 0.256-0.379 mmol/L. In the present invention, the salty threshold of the ASAEAQMWR was 0.0625 mg/mL (i.e. 0.06 mmol/L), and the salty threshold of the ALFDR was 0.125 mg/mL (i.e. 0.201 mmol/L). The ASAEAQMWR had a better saltiness-enhancing effect. It can be seen that the computer simulation and prediction method of the present invention can effectively identify and predict umami peptides, and the umami peptides and salty peptides in the present invention had broad prospects for application in seasonings.

### Example 8 Molecular Docking Between Umami Peptides and T1R1/T1R3 Receptors

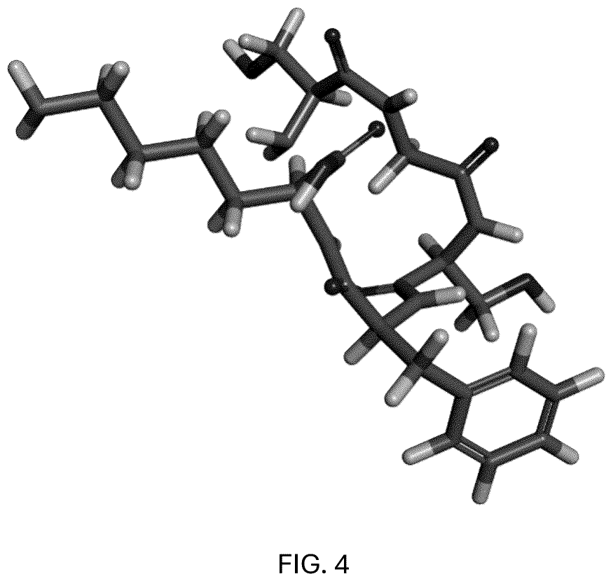

A molecular structural formula of the umami peptide SGSFK was drawn by using a KingDraw 20.0 software, energy of the umami peptide was minimized by using the KingDraw 3D 20.0, and a 3D structure of the umami peptide SGSFK was obtained by using a Discovery Studio 4.5 Client. The 3D structure of the umami peptide SGSFK was shown in FIG. 4.

A three-dimensional (3D) structure of the umami receptor T1R1/T1R3 was established by searching sequences of T1R1/T1R3 (T1R1: Q7RTX1; T1R3: Q7RTX0) in a UniProtKB database.

The URL of the UniProtKB database is https://www.uniprot.org.

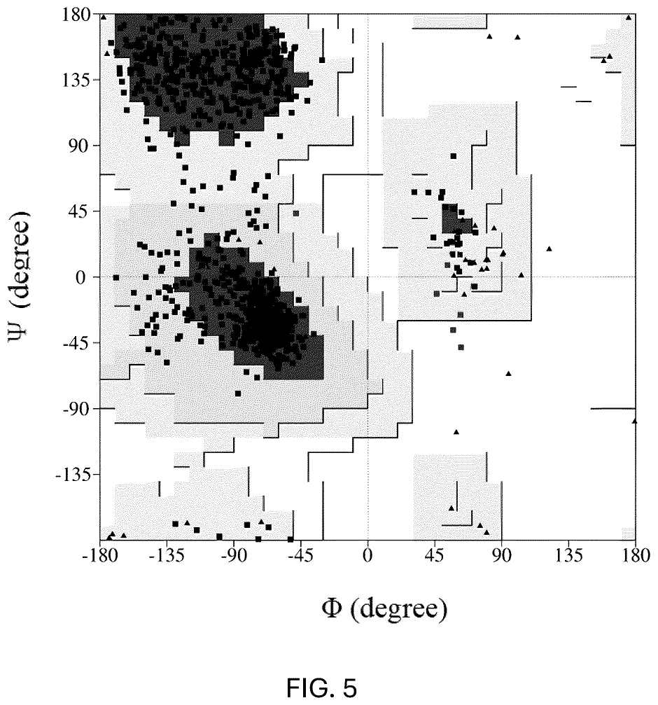

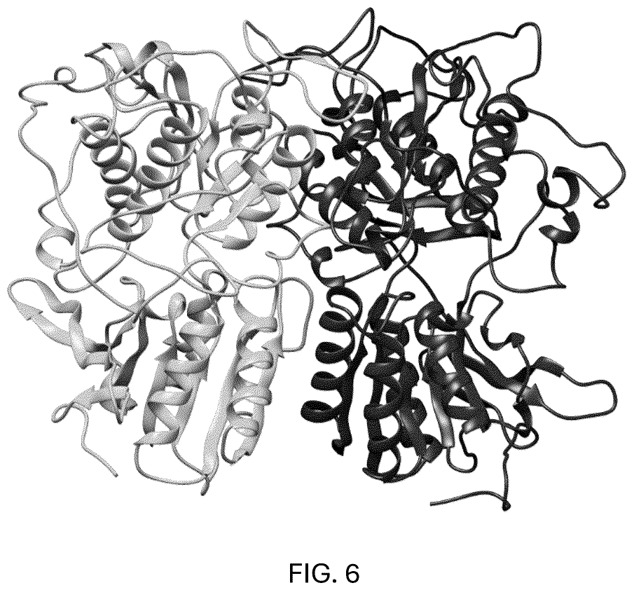

A SWISS-MODEL online modeling tool was used to sequentially add the sequences of T1R1/T1R3, and 5X2m was used as a template to establish a PDB homology model. Then, the preliminary homology model was loaded into a GROMACS 2023 for dynamics optimization, and the rationality of the model structure was evaluated by a ramachandran plot. The ramachandran plot of the umami receptor T1R1/T1R3 was shown in FIG. 5, and the constructed T1R1/T1R3 receptor has a relatively stable structure. A structural model of the umami receptor T1R1/T1R3 was shown in FIG. 6.

The URL of the SWISS-MODEL online modeling tool is https://swissmodel.expasy.org.

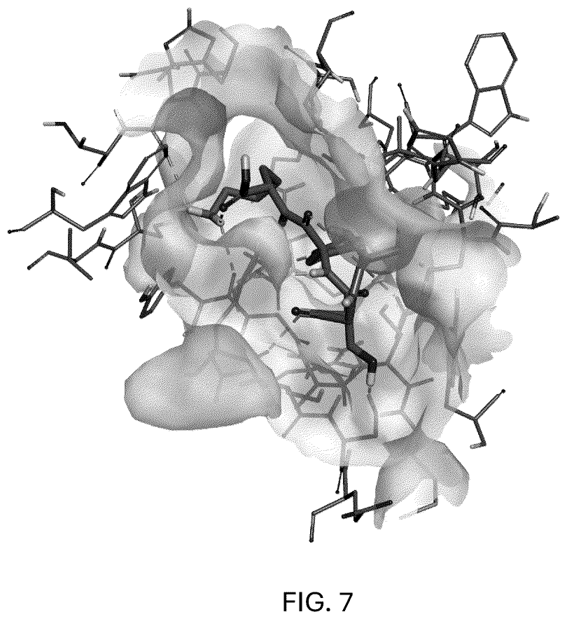

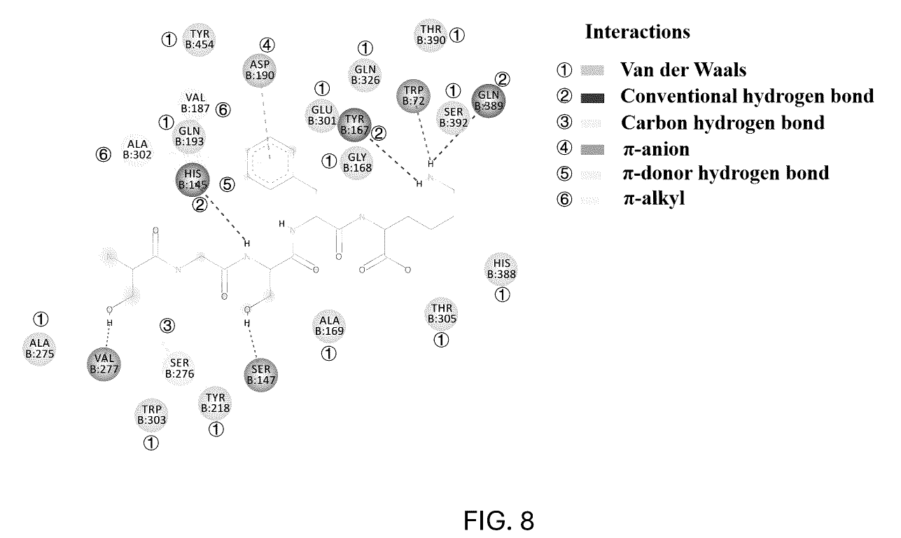

Molecular docking: Autdock 1.5.7 was used to dehydrate and hydrogenate the receptor and calculate the gasteiger charge. Finally, all atoms were assigned to an AD4 type and saved in a PDBQT format. The processed umami receptor T1R1/T1R3 was molecularly docked with the umami peptide SGSFK by using an Autodock Vina algorithm in PyRx, so as to simulate an interaction between a ligand and a receptor. A schematic diagram of the molecular docking between the umami receptor T1R1/T1R3 and the umami peptide SGSFK was shown in FIG. 7. A central site of the umami receptor T1R1/T1R3 binding state was predicted in DeepSite, a central coordinate of a docking box was set to (70, 53, 46) (x, y, z), the box size was set to 30×30×30, and the number of dockings was set to 8 times; and the umami receptor T1R1/T1R3 was docked with the umami peptide SGSFK to obtain a Vina score of the peptide. The obtained docking energy between the umami peptide SGSFK and the umami receptor T1R1/T1R3 was −7.6 kcal/mol, and the peptide was bound to a T1R3 receptor of a dimer. The interaction force and action site between the ligand and the receptor of the peptide were analyzed by using a Discovery Studio 4.5 Visualizer. A schematic diagram of a receptor-ligand interaction force between an active site of the umami receptor T1R1/T1R3 and the umami peptide SGSFK was shown in FIG. 8. Previous studies have shown that the binding of the umami peptides to a T1R3 subunit is mainly through hydrogen bonds and hydrophobic interaction. In the present invention, there are 6 types of interaction forces between the umami peptide SGSFK and the T1R3 subunit, including van der waals, conventional hydrogen bond, carbon hydrogen bond, π-anion, π-donor hydrogen bond, and π-alkyl. The binding of the umami peptide SGSFK to the T1R3 subunit is mainly through the interaction between the van der waals force and the hydrogen bond. A total of 23 amino acid residues in the T1R3 subunit play a role in forming an interaction with the umami peptide SGSFK, including Ala 275, Val 277, Trp 303, Ser 276, Tyr 218, Ser 147, Ala 169, Thr 305, His 388, His 145, Ala 302, Gln 193, Val 187, Tyr 454, Asp 190, Glu 301, Tyr 167, Gly 168, Gln 326, Trp 72, Thr 390, Ser 392, and Gln 389. Among them, Ser 276, Gln 389, Tyr 167, His 145, Asp 190, Val 187, and Ala 302 amino acid residues are mainly connected to the umami peptides through the conventional hydrogen bond, π-anion, and π-alkyl.

The above examples are provided to those skilled in the art to fully disclose and describe how to implement and use the claimed embodiments, and are not intended to limit the scope of the present invention herein. Modifications that are obvious to those skilled in the art will fall within the scope of the appended claims.

What is claimed is:

1. An application of an umami peptide derived from Portunus trituberculatus as or in preparation of an umami agent, wherein an amino acid sequence of the umami peptide derived from the Portunus trituberculatus is SGSFK, as shown in SEQ ID NO. 4.
